(19) United States
(12) Patent Application Publication
(10) Pub. No.: US 2023/0190187 A1
(43) Pub. Date: Jun. 22, 2023

(54) DEVICE, METHOD, PROGRAM, AND SYSTEM FOR DETERMINING DEGREE OF PROGRESSION OF DISEASE

(71) Applicant: CureApp, Inc., Tokyo (JP)

(72) Inventor: Kohta Satake, Chuo-ku, Tokyo (JP)

(73) Assignee: CureApp, Inc., Tokyo (JP)

(21) Appl. No.: 17/926,332

(22) PCT Filed: May 19, 2021
(86) PCT No.: PCT/JP2021/018908

§ 371 Date: Dec. 20, 2022

(30) Foreign Application Priority Data
May 19, 2020 (JP) 2020-087192

(51) Int. Cl. A61B 5/00; A61B 5/11; A61B 5/145

(52) U.S. Cl. CPC A61B 5/4842; A61B 5/1118; A61B 5/14542

(57) ABSTRACT

A device for measuring a degree of progression of disease includes a processor that: acquires a continuously measured arterial oxygen saturation degree, determines a reduction-related index related to reduction by exercise load of the measured arterial oxygen saturation degree based on the continuously measured arterial oxygen saturation degree, acquires information indicating a magnitude of the exercise load, and determines the degree of progression of the disease based on the information indicating the magnitude of the exercise load and the determined reduction-related index.

### TECHNICAL FIELD

The present invention relates to devices, methods, programs and systems for determining the degree of progression of a disease.

### BACKGROUND ART

Although various respiratory/circulatory related diseases are treated at clinical sites (Patent Document 1), there is a case where respiratory diseases such as interstitial pneumonia, viral pneumonia, and COPD are partially rapidly exacerbated while the progress is being observed at home by oral prescription after diagnosis in a hospital. Under the circumstances, a patient visits the hospital due to worsening of subjective symptoms such as cough, sputum, and respiratory discomfort, and through examinations of SpO₂/Xp/CT/blood collection and the like, such exacerbations are diagnosed, which may be followed by treatment enhancement due to hospitalization, or in some cases, it may become severe and intensive treatment such as artificial respiratory management may become necessary.

### PRIOR ART DOCUMENT LIST

### Patent Literature

Patent Document 1: JP 2011-12796 A

### SUMMARY OF INVENTION

### Technical Problem

It is considered possible to contribute to improvement of fatality without requiring hospital treatment or ventilator management by finding such exacerbation at an early stage and performing additional treatment at an early stage, but there is a problem that a doctor and a patient cannot find a sign of exacerbation at an early stage before the subjective symptoms such as cough, sputum, and respiratory discomfort are exacerbated as the exacerbation symptom, and thus the patient may not receive an examination in a hospital until the disease progresses and a serious subjective symptom appears.

An object of the present invention is to provide a device, a system, a method, and a program for determining the degree of progression of a disease.

### Solution to Problem

The present invention has been made in view of the problems described above, and has characteristics such as the following. That is, a device according to one embodiment of the present invention is a device for measuring a degree of progression of a disease, the device being configured to acquire a continuously measured arterial oxygen saturation degree; determine a reduction-related index related to reduction by exercise load of the measured arterial oxygen saturation degree based on the continuously measured arterial oxygen saturation degree; acquire information indicating a magnitude of the exercise load; and determine the degree of progression of the disease based on the information indicating the magnitude of the exercise load and the determined reduction-related index.

Furthermore, the reduction-related index may include a reduction integrated value based on a time integration of the arterial oxygen saturation degree from start of reduction of the arterial oxygen saturation degree to a steady state at the time of load; determining the reduction-related index may include determining a reduction integrated value of the measured arterial oxygen saturation degree based on the continuously measured arterial oxygen saturation degree; and the determination may include determining a degree of progression of a respiratory/circulatory disease based on the information indicating the magnitude of the exercise load and the determined reduction integrated value.

The determination may include determining a reduction integrated boundary threshold value and a reduction integrated exacerbation threshold value smaller than the reduction integrated boundary threshold value based on the information indicating the magnitude of the exercise load; and determination may be made as a boundary state when the determined reduction integrated value is smaller than or equal to the reduction integrated boundary threshold value and greater than the reduction integrated exacerbation threshold value, and as an exacerbation state when the determined reduction integrated value is smaller than or equal to the reduction integrated exacerbation threshold value.

After determining as the boundary state in the determination, the device may acquire a newly measured arterial oxygen saturation degree, re-execute the determination based on the acquired arterial oxygen saturation degree, and determine as the exacerbation state when the determination as the boundary state is repeated for greater than or equal to a predetermined number of times.

The reduction-related index may include a reduction start required time from start of a load state by exercise until start of reduction of the arterial oxygen saturation degree; the determination of the reduction-related index may include acquiring information indicating timing at which the load state by exercise has started, and determining a reduction start required time of the measured arterial oxygen saturation degree based on the timing at which the load state has started and the continuously measured arterial oxygen saturation degree; and the determination may include determining the degree of progression of the disease based on the information indicating the magnitude of the exercise load and the determined reduction start required time.

The reduction-related index may include a load steady saturation degree that is an arterial oxygen saturation degree when a steady state is obtained after reduction of the arterial oxygen saturation degree has started; the determination of the reduction-related index may include determining the load steady saturation degree based on the continuously measured arterial oxygen saturation degree; and the determination may include determining the degree of progression of the disease based on the information indicating the magnitude of the exercise load and the determined load steady saturation degree.

The device may present warning information based on the determined degree of progression of the respiratory disease.

The device may continuously measure and acquire arterial oxygen saturation degree.

The device may detect the movement of the user's body to generate and acquire information indicating the magnitude of the exercise load.

A method according to one embodiment of the present invention is a method for determining degree of progression of a disease, the method causing a computer to execute steps of acquiring a continuously measured arterial oxygen saturation degree; determining a reduction-related index related to reduction by exercise load of the measured arterial oxygen saturation degree based on the continuously measured arterial oxygen saturation degree; acquiring information indicating a magnitude of the exercise load; and determining the degree of progression of the disease based on the information indicating the magnitude of the exercise load and the determined reduction-related index.

A program according to one embodiment of the present invention may be a program for causing a computer to execute the method described above.

A system according to one embodiment of the present invention is a system for determining degree of progression of a disease, the system including continuously measuring arterial oxygen saturation degree; determining a reduction-related index related to reduction by exercise load of the measured arterial oxygen saturation degree based on the continuously measured arterial oxygen saturation degree; acquiring information indicating a magnitude of the exercise load; and determining the degree of progression of the disease based on the information indicating the magnitude of the exercise load and the determined reduction-related index.

### Advantageous Effects of Invention

Through the use of the present invention, a device, a system, a method and a program for determining a degree of progression of a disease can be provided.

### BRIEF DESCRIPTION OF DRAWINGS

FIG. 1 is a configuration diagram of a system according to one embodiment of the present invention.

FIG. 2 is a hardware configuration diagram of an arterial oxygen saturation degree measuring instrument and a user device according to one embodiment of the present invention.

FIG. 3 is a flowchart according to one embodiment of the present invention.

FIG. 4 is a diagram illustrating temporal transition of arterial oxygen saturation degree as a reference index according to one embodiment of the present invention.

FIG. 5 is a flowchart for correcting determination according to one embodiment of the present invention.

FIG. 6 is a view illustrating a reduction integrated value of a reference index according to one embodiment of the present invention.

FIG. 7 is a flowchart when the reduction integrated value according to one embodiment of the present invention is used.

FIG. 8 is a view illustrating a reduction integrated value of the determination target according to one embodiment of the present invention.

FIG. 9 is a flowchart when the reduction start required time according to one embodiment of the present invention is used.

FIG. 10 is a view illustrating a reduction start required time according to one embodiment of the present invention.

FIG. 11 is a flowchart when the load steady saturation degree according to one embodiment of the present invention is used.

FIG. 12 is a view illustrating a load steady saturation degree according to one embodiment of the present invention.

### DESCRIPTION OF EMBODIMENTS

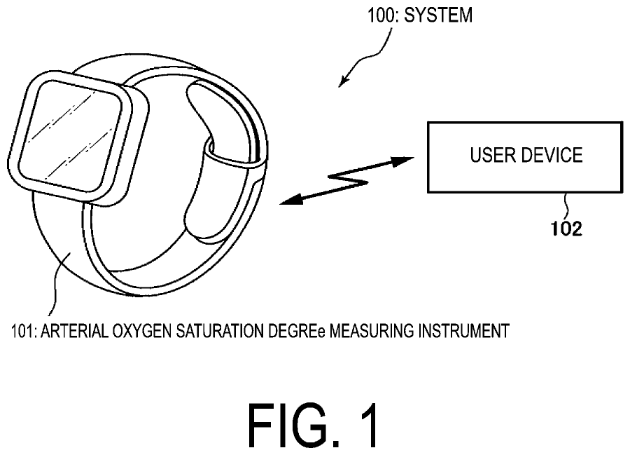

FIG. 1 is a configuration diagram of a system according to one embodiment of the present invention. A system 100 is used to determine the degree of progression of a disease such as a respiratory disease or circulatory disease (respiratory/circulatory disease) that causes hypoxemia with circulating breathing dynamics abnormality, such as pneumonia, COPD, chronic respiratory failure and chronic heart failure, and includes an arterial oxygen saturation degree measuring instrument 101 and a user device 102 used by a user, who is a person to be measured. The arterial oxygen saturation degree measuring instrument 101 and the user device 102 are connected by wired communication or wireless communication.

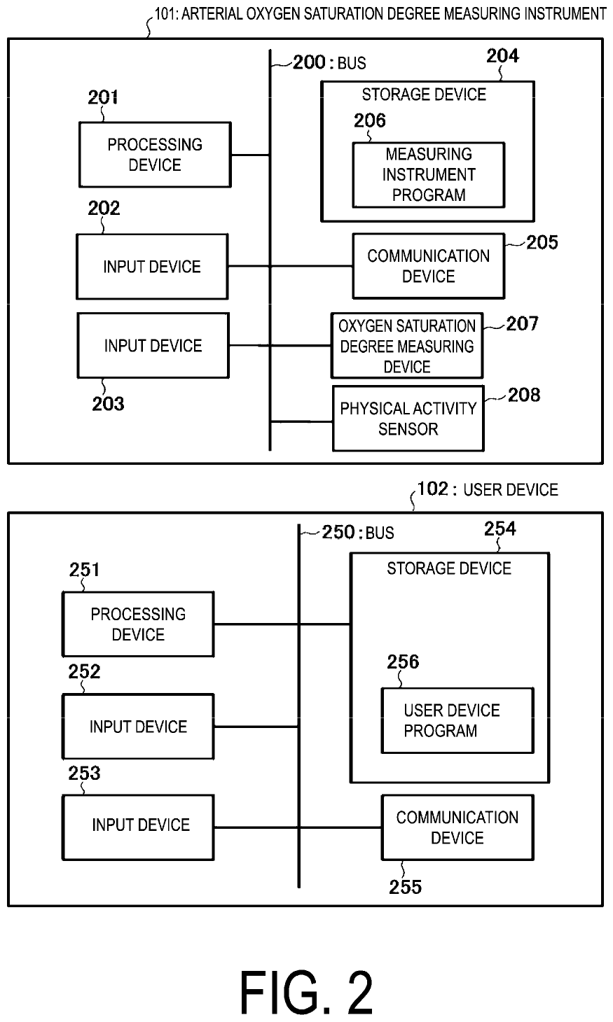

FIG. 2 illustrates an example of a hardware configuration diagram of the arterial oxygen saturation degree measuring instrument 101 and the user device 102. In the present embodiment, the arterial oxygen saturation measuring instrument 101 and the user device 102 are electronic devices each including a processing device 201, 251, an output device 202, 252, an input device 203, 253, a storage device 204, 254, and a communication device 205, 255. The arterial oxygen saturation degree measuring instrument 101 includes an arterial oxygen saturation degree measuring device 207 and a physical activity sensor 208. Each of these configuring devices is connected via a bus 200, 250, but a mode in which each of the configuring devices are individually connected as needed may be adopted. Programs 206 and 256 are stored in the storage devices 204 and 254. The program may be referred to as an app.

Each of the processing devices 201, 251 performs various processes based on the programs 206, 256, input data from the input devices 203, 253 or data received from the communication devices 205, 255. A processor that controls each device included in each of the processing devices 201, 251, the arterial oxygen saturation degree measuring instrument 101, and the user device 102 is provided, and various processes are performed with registers and the storage devices 204, 254 included in the processor as work regions.

The output devices 202, 252 output display and audio of the screen in accordance with the control of the processing devices 201, 251. The input devices 203, 253 have a function of accepting input from a user, such as a keyboard, a touch panel, a touch pad, an input button, or the like.

The storage devices 204, 254 include a main memory, a buffer memory, and a storage, and are storage devices provided in a general computer such as a storage device using a flash memory such as a RAM that is a volatile memory and an eMMC, a UFS, or an SSD that is a nonvolatile memory, a magnetic storage device, or the like. The storage device 204, 254 may also include an external memory. The communication device 205, 255 performs wired communication using an Ethernet (registered trademark) cable or the like, or wireless communication using Bluetooth (registered trademark) and a wireless LAN, or the like, and enables communication to be performed between the arterial oxygen saturation degree measuring instrument 101 and the user device 102.

The arterial oxygen saturation degree measuring device 207 is a pulse oximeter that measures percutaneous arterial oxygen saturation degree (SpO₂) in the present embodiment, but may be any device as long as it measures the arterial oxygen saturation degree of the user.

The physical activity sensor 208 is one that uses at least one of a gyroscopic sensor, an acceleration sensor, an orientation sensor, and a GPS sensor to detect the movement of the user's body and generate information indicating the state of the user's physical activity. Here, the arterial oxygen saturation degree measuring instrument 101 can be attached to the user, and the SpO₂ can be measured and the state of physical activity of the user can be measured while exercising.

The physical activity information indicating the state of physical activity of the user includes information indicating the information indicating the movement of the user's body along with time information. Therefore, the physical activity information may indicate the timing at which the user has started the exercise, and can further indicate the magnitude of the exercise load. Here, the physical activity sensor 208 estimates the exercise intensity (METs) as a magnitude of the exercise load associated with time information based on the detected movement of the user's body. For example, which state of (i) walking, (ii) bicycle, fast walking, (iii) going up the staircase, jogging, (iv) running, carrying of heavy luggage is estimated, and based on each estimated physical activity state, (i) walking=3 METs, (ii) bicycle, fast walking=4.5 METs, (iii) going up the staircase, jogging=6 METs, (iv) running, carrying of heavy luggage=8 METs and exercise load are estimated.

A typical technique for detecting the movement of a user's body and estimating the exercise intensity can be used. Rather than the value of such discrete exercise load, the value of the continuous exercise load may be estimated based on the physical activity information. In addition, the magnitude of the exercise load is not limited to the exercise intensity, and any index can be adopted as long as it can indicate the magnitude of the exercise load.

In the present embodiment, the physical activity information including the exercise load associated with the time information generated by the physical activity sensor 208 is transmitted to the user device 102, but the physical activity sensor 208 may transmit the information indicating the detected movement of the user's body to the user device 102 together with the time information, and determine the start timing of the exercise and the magnitude of the exercise load based on the information received by the user device 102.

Furthermore, the arterial oxygen saturation degree measuring instrument 101 may be configured to not include the physical activity sensor 208. In this case, as the user inputs the time at which the physical activity started and the exercise load through the arterial oxygen saturation degree measuring instrument 101 or the user interface of the user device 102, information indicating the timing at which the user device 102 has started the physical activity and the magnitude of the exercise load can be acquired. Furthermore, a predefined exercise load may be stored in the user device 102 for the exercise load, and this may be used.

In the present embodiment, the respective programs are executed in the processing device illustrated in FIG. 2, and the functions described below are executed by operating in cooperation with the respective pieces of hardware, but can also be realized by hardware by configuring an electronic circuit or the like for realizing the respective functions.

In the present embodiment, the arterial oxygen saturation degree measuring instrument 101 is used as a smart watch including, for example, a physical activity sensor, but may be a pulse oximeter in which the physical activity information is manually input by the user as described above, and only the measurement of SpO₂ is performed. The user device 102 is a smartphone, but may be a desktop computer or a notebook computer, or may be a portable information terminal, a mobile phone, or a tablet terminal. The arterial oxygen saturation degree measuring instrument 101 and the user device 102 are configured to be wirelessly connected by Bluetooth (registered trademark).

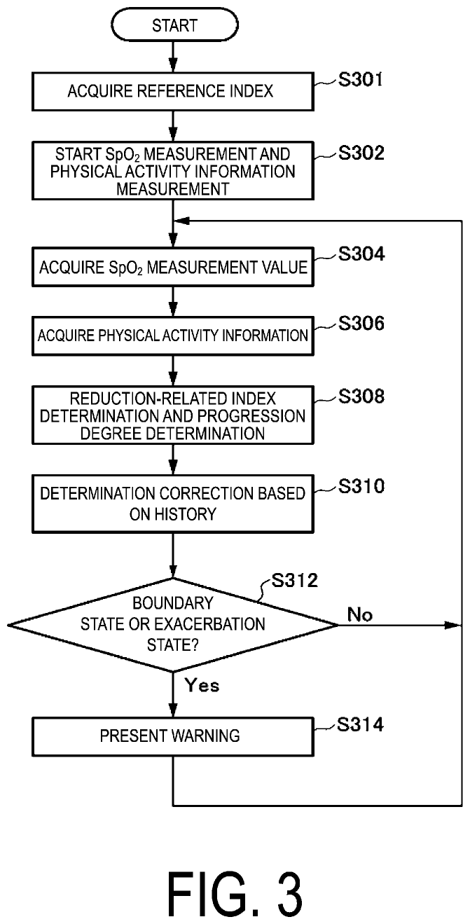

Next, the operation of the system in the present embodiment is described with reference to FIG. 3. The user measures the SpO₂ for the reference index. For example, when a user suffering from respiratory/circulatory disease receives the physician's examination, and the symptoms are stable at that time point, determination is made to perform home care. In addition, a smart watch, which is the arterial oxygen saturation degree measuring instrument 101, is worn at the time of examination according to the instruction of the doctor, walking is performed for a predetermined time (e.g., 30 minutes), and the physical activity information and the SpO₂ during that time are continuously measured. Furthermore, a respiratory/circulatory disease progression degree determination app for carrying out the present invention is installed in the user's smartphone 102, the measured SpO₂ and physical activity information is received from the smart watch, and the measured SpO₂ is stored as a reference index for the exercise intensity indicated by the physical activity information (S301).

The SpO₂ measured at the time of examination of a doctor is not necessarily a measurement value of a non-disabled state, but is a measurement value of when determined as a stable state by the doctor, and thus it can be used as a reference. Instead of measuring at the time of examination of the doctor, the smart watch 101 equipped with the pulse oximeter may be worn and used regularly, and the exercise intensity and the SpO₂ measured during that time can be used as a reference index. The measurement value acquired from normal life is considered to be a measurement value in a non-disabled state, and thus can be used as a reference index.

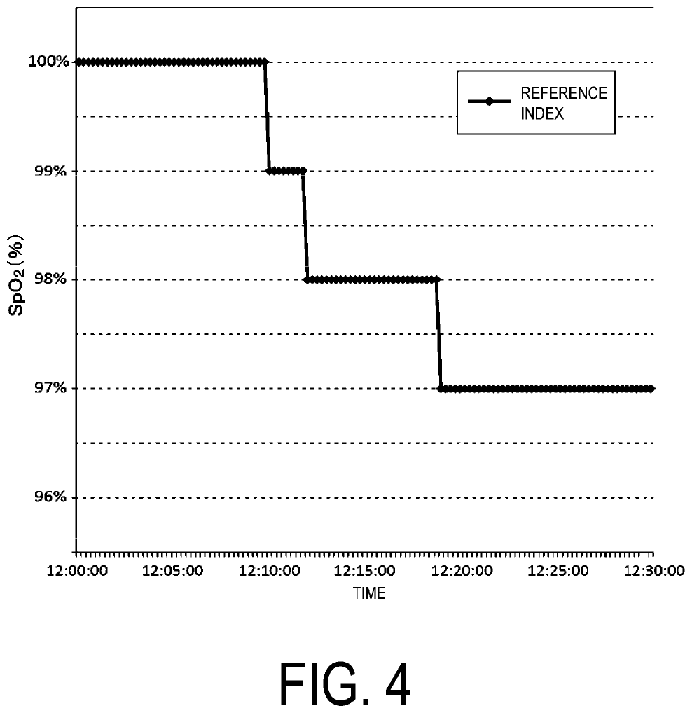

One example of data that serves as a reference index is shown in Table 1 and FIG. 4.

TABLE 1

| Time (seconds) | SpO₂ | METs |
|---|---|---|
| 11:59:45 | 100% | 1 |
| 12:00:00 | 100% | 3 |
| 12:00:15 | 100% | 3 |
| 12:00:30 | 100% | 3 |
| . . . | . . . | . . . |
| 12:09:45 | 100% | 3 |
| 12:10:00 | 99% | 3 |
| 12:10:15 | 99% | 3 |
| . . . | . . . | . . . |
| 12:11:45 | 99% | 3 |
| 12:12:00 | 98% | 3 |
| . . . | . . . | . . . |
| 12:18:45 | 98% | 3 |
| 12:19:00 | 97% | 3 |
| . . . | . . . | . . . |
| 12:30:00 | 97% | 3 |

Table 1 and FIG. 4 show SpO₂ and exercise intensity measured during 30 minutes of walking while wearing a smart watch equipped with a pulse oximeter. It is shown that walking has started as METs changed from 1 (resting) to 3 (walking) at 12:00:00. It is shown that SpO₂, which was 100%, is reduced to 99% at 12:10:00 due to the exercise load by walking, then reduced to 97% at 12:19:00, and a steady state is obtained at 97%. The arterial oxygen saturation degree in the load steady state is referred to as a load steady saturation degree.

The fluctuation of SpO₂ is monitored for a predetermined period to determine a steady state, and determination is made as a steady state when the fluctuation of greater than or equal to a predetermined percentage is continued for longer than or equal to a constant period, and the average value of the SpO₂ of the constant period may be set as the load steady saturation degree. Here, in a case where a state in which the fluctuation rate with respect to the average value of one minute is within 5% (e.g., the average value is 97%, the fluctuation range is within 96.515 to 97.485%) is continued for ten minutes, it is referred to as a steady state, and the average value of such ten minutes is referred to as the load steady saturation degree. SpO₂ when determined to be the steady state by other criteria may be the load steady saturation degree.

Thereafter, continuous measurement of the SpO₂ and the physical activity information for the respiratory/circulatory disease progression degree determination is started using the smart watch 101 (S302). The continuous measurement may be performed constantly while wearing the smart watch serving as the arterial oxygen saturation degree measuring instrument 101, or may be manually started by wearing the arterial oxygen saturation degree measuring instrument 101 immediately before starting the exercise. For example, walking may be performed several times a day at defined times, and the continuous measurement may be started immediately before the start of walking.

The smart watch 101 transmits the measured SpO₂ at predetermined intervals, and the user device 102, which is a smartphone, receives and acquires the same (S304). Furthermore, the smart watch 101 also transmits the physical activity information including the information indicating the measured exercise intensity at predetermined intervals, and the user device 102, which is a smartphone, receives and acquires the same (S306). It is assumed that the information indicating the exercise intensity and the SpO₂ are associated with time information, and indicate when the estimated value or the measurement value is obtained. The measurement value of SpO₂ and the physical activity information may be collectively transmitted and received as one information.

The user device 102 determines a reduction-related index related to the reduction by the exercise load of the measured SpO₂ based on the acquired measurement value of the SpO₂, and determines the degree of progression of the disease based on the information indicative of the magnitude of the exercise load and the determined reduction-related index (S308).

The reduction-related index related to the reduction of SpO₂ due to the exercise load is an index indicating a mode of reduction, such as manner and extent of reduction when SpO₂ is reduced by the exercise load, and can be, for example, (i) a reduction integrated value determined based on time integration of the arterial oxygen saturation degree from the start of reduction of the arterial oxygen saturation degree to a load steady state, (ii) a reduction start required time from the start of the load state by the exercise to the start of reduction of the arterial oxygen saturation degree, and (iii) a load steady saturation degree, which is the arterial oxygen saturation degree in the steady state after the start of reduction of the arterial oxygen saturation degree.

The present invention can also be carried out using only one reduction-related index, and can also be carried out using two or more reduction-related indices. For example, the process may be executed in parallel or in series for three factors, the reduction integrated value, the reduction start required time and the load steady saturation degree, as the reduction-related index, and the most severe determination result of the respective determination results can be determined as final determination result.

Next, the user device 102 corrects the determination result in S308 based on the history of the previous determination result (S310). For example, if continuously determined as the boundary state, the determination can be corrected as the exacerbation state. The previous determination result is stored in the storage device 254 to correct the determination result based on the history of the determination result. Here, the stored determination result is the corrected determination result.

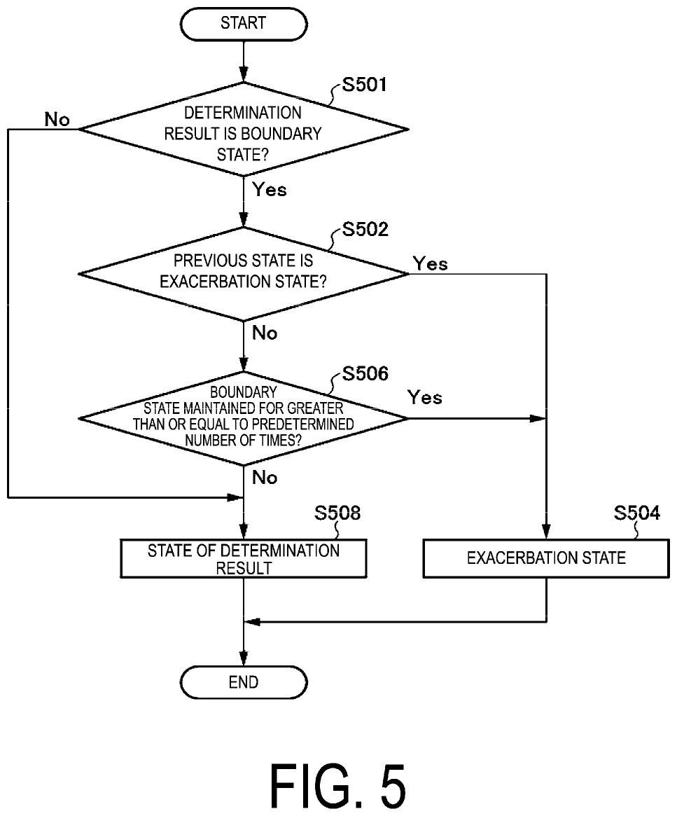

An example of a correction process of the determination result will be described based on a processing flow illustrated in FIG. 5. In the present embodiment, the state to be determined is three states of the stable state, the boundary state, and the exacerbation state, but may be only two states of the stable state and the exacerbation state, or may be four or more states. The stable state is a state in which the respiratory disease or the circulatory disease is stable and is not worsening, the boundary state is a state in which the possibility of the respiratory disease or the circulatory disease worsening cannot be denied but it cannot be said that the respiratory disease or the circulatory disease is worsening, and the exacerbation state is a state in which the possibility of the respiratory disease or the circulatory disease worsening is high and close examination and treatment at a medical institution is required.

Determination is made on whether or not the determination result in S308 is a boundary state (S501). If in the boundary state, determination is made on whether or not the previous determination result has been the exacerbation state (S502). If the previous determination result has been the exacerbation state, the determination result is corrected to the exacerbation state even if the present determination result is the boundary condition (S504). The exacerbation state is a serious state that requires urgent medical examination by the doctor, and if determination is made even once as the exacerbation state, it is not preferred to determine that the exacerbation state has been resolved without diagnosis by the doctor.

If the previous state is not the exacerbation state, determination is made on whether or not the boundary state is maintained for greater than or equal to a predetermined number of times based on the history of the previous determination results (S506). If the boundary state is maintained for greater than or equal to a predetermined number of times, the determination result is corrected to the exacerbation state (S504). If the boundary state is not maintained for greater than or equal to a predetermined number of times, the determination result of the boundary state is maintained (S508).

If the determination result is not a boundary condition in S501, the state of the determination result is maintained (S508). That is, in a case where the state is determined to be the stable state or the exacerbation state, the state of the determination result of the stable state or the exacerbation state is maintained, and the determination result is not corrected.

In the present embodiment, the determination result in S308 is corrected based on the history of the previous determination results, but the determination result may be used as is without performing correction based on the history.

Next, the user device 102 determines whether or not the determination result is the boundary state or the exacerbation state (S312). In the case of the boundary state or the exacerbation state, warning information based on the determination result is presented (S314). For example, warning information can be displayed on a display that is the output device 252 of the user device 102, a warning sound can be output by voice, or warning information can be output.

For example, when determined as the boundary state, the warning information “deterioration in blood oxygen saturation degree at the time of load is observed, but the degree of deterioration is not so strong, and it is not a state where exacerbation of pneumonia is positively suspected. The data will be carefully measured in the future, and if there is a sign of exacerbation of pneumonia, this will be notified.” is displayed on the display serving as the output device of the user device 102, and when determined as the exacerbation state, “deterioration in blood oxygen saturation degree at the time of load is observed, and a possibility of exacerbation of pneumonia is suggested. Please visit the hospital and consult the doctor” can be displayed. In addition, for example, warning information may be transmitted to an electronic device (not illustrated) used by a doctor via the Internet, and the warning information may be presented to the doctor.

When the presentation of the warning information is completed, the process returns to S304, the measurement value of SpO₂ is newly acquired, and the processes from S306 to S314 are repeatedly executed. The arterial oxygen saturation degree measuring instrument 101 continuously performs the measurements of SpO₂ and physical activity information even during S304 to S314.

When determined as the exacerbation state, after the warning information is presented, the progression degree determination process may be terminated without returning to S304. When determined as not the boundary state nor the exacerbation state in S312, that is, when determined as the stable state, the process returns to S304 without the warning information being output. When determined as the stable state, information indicating the stable state may be presented to the user.

Next, specific processing of the reduction-related index determination and the progression degree determination (S308) when (i) the reduction integrated value, (ii) the reduction start required time, and (iii) the load steady saturation degree are used as the reduction-related indices will be described.

### [Reduction Integrated Value]

First, an embodiment in a case where a reduction integrated value is used as the reduction-related index will be described. As previously mentioned, the reduction integrated value is determined based on the time integration of arterial oxygen saturation degree from the start of reduction in the arterial oxygen saturation degree until the load steady state.

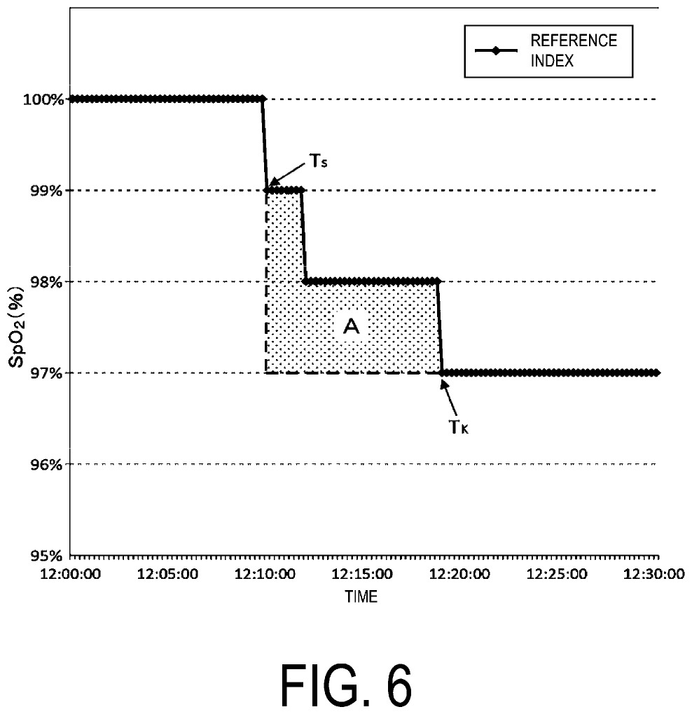

Here, as shown in Equation 1, the reduction integrated value is a time integrated value of a difference between an arterial oxygen saturation degree (SpO₂(t)) and the load steady saturation degree (K) from the time point (Tₛ) at which the reduction of the arterial oxygen saturation degree has started to the time point (T_{K}) at which a load steady state is obtained. For example, the reduction integrated value of the reference index illustrated in FIG. 4 is the area of part A in FIG. 6. Instead of being the difference with the load steady saturation degree (K), it may be a time integrated value of the arterial oxygen saturation degree (SpO₂ (t)) from the time point (Tₛ) at which the reduction of the arterial oxygen saturation degree has started to the time point (T_{K}) at which the load steady state is obtained.

$$I = {\int }_{Ts}^{{T}_{K}} ( {Sp O}_{2} ( t ) - K ) dt \qquad [ EQUATION 1 ]$$

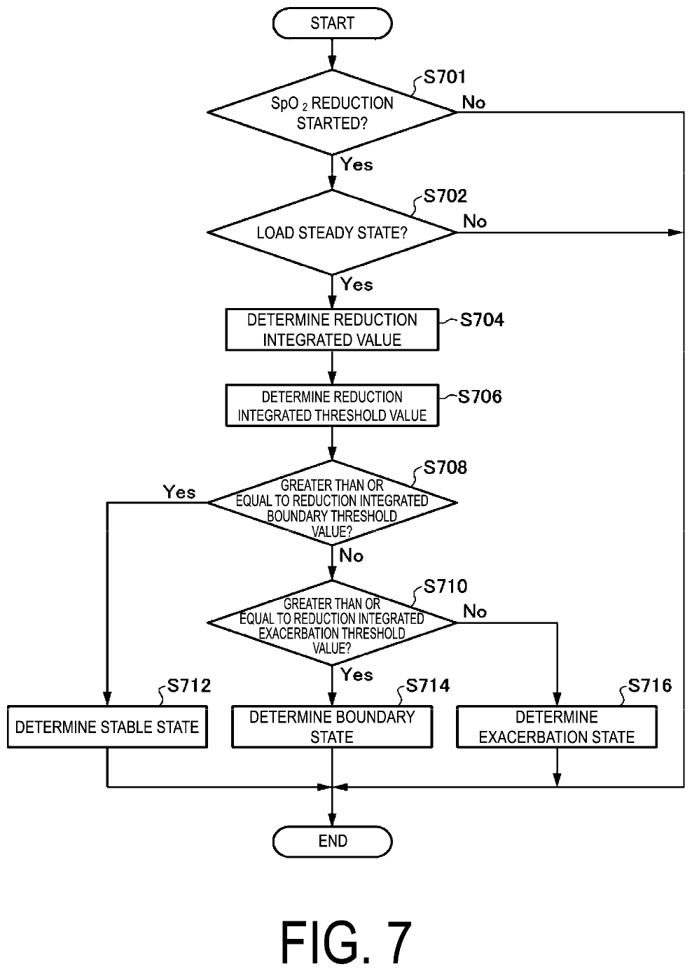

Of the progression degree determination processing flow illustrated in FIG. 3, a specific processing flow is illustrated in FIG. 7 for a case in which the reduction integrated value is used as the reduction-related index in the reduction-related index determination and progression degree determination (S308).

First, whether or not reduction of the SpO₂ has started is determined based on the SpO₂ measurement values previously acquired in S304 (S701). If reduction has not started, the reduction-related index determination and progression degree determination process are terminated, and the process returns to S304. If reduction has started, whether or not SpO₂ is in a load steady state is determined (S702). If not in the steady state, the reduction-related index determination and progression degree determination process are terminated, and the process returns to S304.

The correction process (S310) may not be executed if the determination result is not output in S308, or determination may be made that the determination result is not the boundary state (S501), and the process may be terminated while maintaining the state of the determination result (S508). Thereafter, since the state is not the boundary state nor the exacerbation state in S312, the process returns to S304.

If in the steady state, the reduction integrated value is determined (S704). More specifically, based on the acquired SpO₂ measurement value, the timing (Tₛ) at which the reduction of the SpO₂ has started, the timing (T_{K}) at which the SpO₂ becomes the load steady state, and the load steady saturation degree (K) are determined, and the reduction integrated value is calculated according to the above Equation 1.

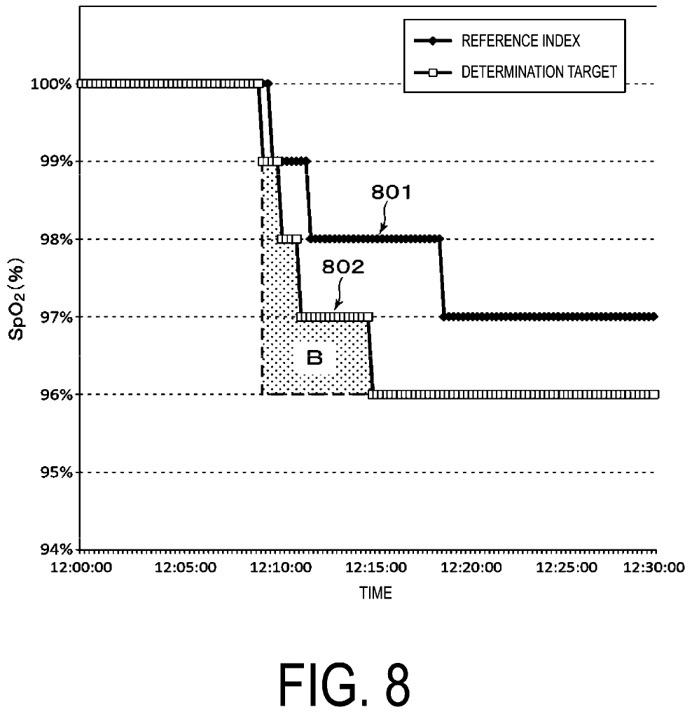

An example of the SpO₂ measurement value and the exercise load to be determined in the degree of progression of the disease is shown in Table 2 and FIG. 8. In FIG. 8, line 801 indicates the transition of SpO₂ of the reference index, and line 802 indicates the transition of the measured SpO₂ to be determined.

TABLE 2

| Time | SpO₂ | METs |
|---|---|---|
| 11:59:45 | 100% | 1 |
| 12:00:00 | 100% | 3 |
| . . . | . . . | . . . |
| 12:09:15 | 100% | 3 |
| 12:09:30 | 99% | 3 |
| . . . | . . . | . . . |
| 12:10:15 | 99% | 3 |
| 12:10:30 | 98% | 3 |
| . . . | . . . | . . . |
| 12:11:15 | 98% | 3 |
| 12:11:30 | 97% | 3 |
| . . . | . . . | . . . |
| 12:15:00 | 97% | 3 |
| 12:15:15 | 96% | 3 |
| . . . | . . . | . . . |
| 12:30:00 | 96% | 3 |

In the measurement value of the determination target, it can be seen that the walking starts from 12:00:00, SpO₂ becomes 99% at 12:09:30, and SpO₂ becomes the steady state (96%) at 12:15:15. Therefore, the reduction start timing (Tₛ) is 12:09:30, the timing (T_{K}) at which the load steady state is obtained is 12:15:15, and the load steady saturation degree (K) is 96%. The reduction integrated value of SpO₂ measured at this time is the area of part B shown in FIG. 8.

Note that the reduction start timing is the first timing of reduction from 100% in the present embodiment, but a timing (12:09:15) one before the timing of reduction from 100% may be the start point, or timing of reduction by greater than or equal to a predetermined value from 100% may be adopted. Any timing may be used as long as the timing indicates that the arterial oxygen saturation degree has reduced.

The user device 102 then determines a reduction integrated threshold value (S706). The reduction integrated threshold value is a threshold value for determining the measured SpO₂ to be determined as a stable state, a boundary state, and an exacerbation state. Here, a reduction integrated boundary threshold value and a reduction integrated exacerbation threshold value smaller than the reduction integrated boundary threshold value are determined. Determination is made as the stable state when the determined reduction integrated value is greater than or equal to the reduction integrated boundary threshold value, determination is made as the boundary state when the determined reduction integrated value is smaller than the reduction integrated boundary threshold value and greater than or equal to the reduction integrated exacerbation threshold value, and determination is made as the exacerbation state when the determined reduction integrated value is smaller than the reduction integrated exacerbation threshold value.

The reduction integrated boundary threshold value and the reduction integrated exacerbation threshold value can be determined as a function of exercise intensity, and in this embodiment are calculated by the following Equations 2 and 3, but this is not the sole case. The load METs is the METs of the determination target.

reduction integrated boundary threshold value=reference reduction integrated value×0.85 [Equation 2]

reduction integrated exacerbation threshold value=reference reduction integrated value×(100−(15+2 log)₂ (load METs)/100 [Equation 3]

The reference reduction integrated value is a reduction integrated value that becomes a reference index, and can be determined based on the exercise load. In the present embodiment, the exercise load of the determination target is also a walking (3 METs), and the calculated reduction integrated value calculated based on the measurement value measured by actually performing walking during examination by a doctor in S301 is used as the reference reduction integrated value. For jogging or the like as well, actual measurement may be performed to store a reference index corresponding to the exercise load, and the reference index may be selected according to the exercise load of the determination target. The reference reduction integrated value may be determined based on data such as gender, age, and weight of the user regardless of the actual measurement value by the user, or a reference reduction integrated value determined in advance for all users may be used.

Furthermore, the reference reduction integrated value for other exercise loads may be calculated by calculation based on the reference reduction integrated value determined for one exercise load. For example, it can be calculated based on the following Equation 4.

reference reduction integrated value (load METs)=reference reduction integrated value (reference METs)×log₂(reference METs)/log₂ (load METs) [Equation 4]

The reference reduction integrated value (reference METs) is, for example, the reference reduction integrated value determined by actually performing walking as described above, and the reference METs is the exercise intensity at this time, and is 3 METs here. The load METs is METs at the time of measurement of the measured arterial oxygen saturation degree to be determined. When the reference reduction integrated value at the time of walking is determined, the reference reduction integrated value for other exercise loads such as jogging can be calculated, and the progression degree determination can be performed based thereon.

Here, since the load METs=3, the measurement value at the time of walking shown in Table 1 is referred to as a reference index. Since the reference index reduction integrated value (A)=660 and the load METs=3, the reduction integrated boundary threshold value=561 and the reduction integrated exacerbation threshold value=540.08 are calculated based on Equations 2 and 3, and the determination target reduction integrated value (B)=525 is calculated.

If the measurement value of the determination target is the measurement data at the time of jogging, the reference reduction integrated value (6 METs)=404.68 can be calculated based on Equation 4, the reference reduction integrated value (A), and the exercise load of jogging (6 METs).

Whether or not the reduction integrated value determined in S704 is greater than or equal to the reduction integrated boundary threshold value is determined (S708), where if true, determination is made as the stable state (S712), and if false, whether or not it is greater than or equal to the reduction integrated exacerbation threshold value is determined (S710). Determination is made as the boundary state if true (S714), and determination is made as the exacerbation state if false (S716). In the present embodiment, the determination target reduction integrated value (B) is less than or equal to the reduction integrated boundary threshold value, and smaller than the reduction integrated exacerbation threshold value, and thus determination is made as the exacerbation state.

Thereafter, re-determination is performed based on the history information in S310. The determination of disease progression degree by the reduction integrated value is performed only once with respect to one exercise from a reduction in arterial oxygen saturation degree due to exercise load until a load steady state is obtained. When a single determination is made, the determination result thereof is stored in the storage device 254 of the user device 102. On the basis of the physical activity information and the measurement value of the arterial oxygen saturation degree, the progression degree determination process is not performed until determination is made that the exercise is once terminated and the arterial oxygen saturation degree has reached the resting steady state, and after determination is made that the resting steady state is reached, the exercise is started again, and the determination of the disease progression degree by the reduction integrated value can be performed. In a case where this is repeated and determination is made as the boundary state at a predetermined number of times, for example, two consecutive times, determination is made as the exacerbation state.

The fact that the reduction integrated value of the determination target is reduced as compared with the reference reduction integrated value indicates that the arterial oxygen saturation degree due to the exercise load reduces in a short time as compared with the reference index, where reduction in the gas exchange preliminary function due to the disease can be detected and the degree of progression of the disease can be determined based on the extent of the reduction.

### Reduction Start Required Time

Next, an embodiment of a case where the reduction start required time is used as the reduction-related index. A portion different from the example of the reduction integrated value will be described in detail, and the description of the similar portion will be omitted. As described above, the reduction start required time is the required time from the start of the load state by exercise until the start of reduction in arterial oxygen saturation degree.

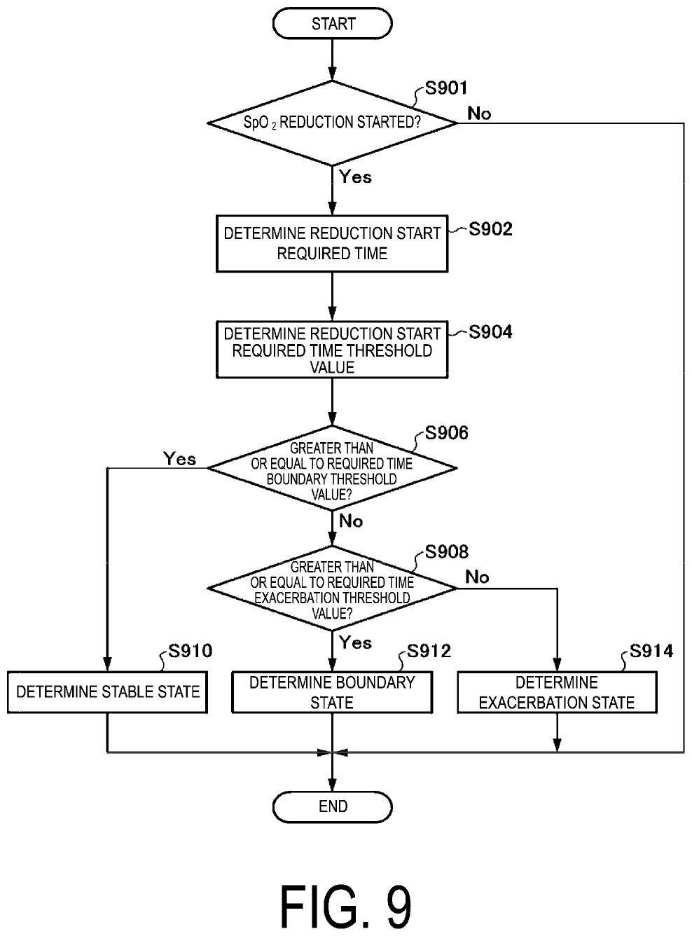

Of the progression degree determination processing flow illustrated in FIG. 3, a specific processing flow is illustrated in FIG. 9 for a case in which the reduction start required time is used as the reduction-related index in the reduction-related index determination and progression degree determination (S308).

First, whether or not reduction of the SpO₂ has started is determined based on the SpO₂ measurement values previously acquired in S304 (S901). If reduction has not started, the reduction-related index determination and progression degree determination process are terminated, and the process returns to S304. If the reduction has started, the reduction start required time of SpO₂ is determined (S902).

The reduction start required time of SpO₂ can be determined by specifying the timing at which the load state due to exercise started and the timing at which reduction of the measured SpO₂ started. That is, the reduction start required time can be calculated by subtracting the time at which the load state due to exercise started from the time at which the reduction of SpO₂ started.

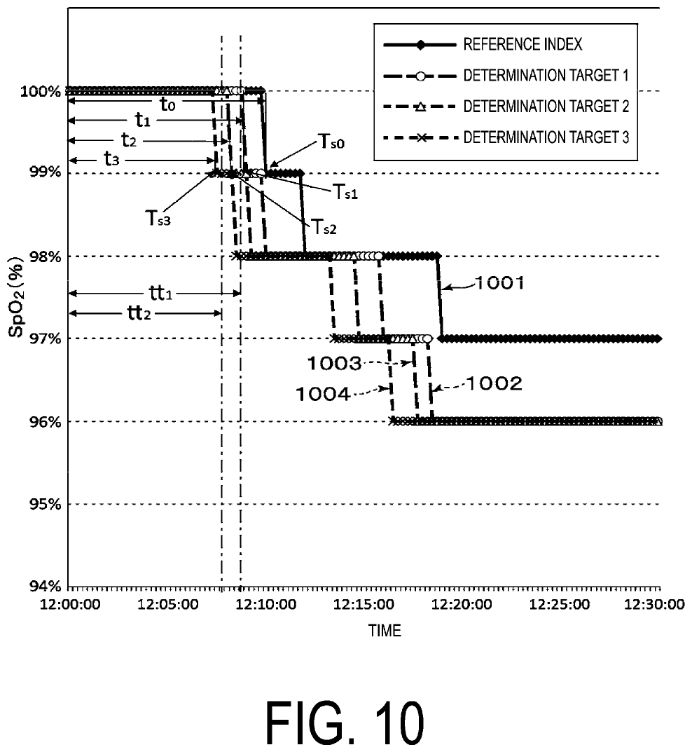

An example of the acquired SpO₂ to be determined is shown in FIG. 10. In FIG. 10, line 1001 indicates the transition of SpO₂ of the reference index, and lines 1002, 1003, 1004 indicate the transition of the measured SpO₂ to be determined.

According to each measurement value of SpO₂ shown in FIG. 10, the reduction is started at the time point of Tₛ₀ (12:10:00) for the reference index, at the time point of Tₛ₁ (12:09:00) for the determination target 1, at the time point of Tₛ₂ (12:08:15) for the determination target 2, and at the time point of Tₛ₃ (12:07:30) for the determination target 3.

In the present embodiment, the physical activity information acquired in S306 is acquired as data associating the information of exercise intensity with the time information as illustrated in Tables 1 and 2, where here, the reference index and the physical activity information of each of the determination targets 1 to 3 all indicate that walking (3 METs) started from 12:00:00. Therefore, the reduction start required times for the reference index and each determination target 1 to 3 are t₀(10:00), t₁(9:00), t₂(8:15) and t₃ (7:30).

Next, the reduction start required time threshold value is determined (S904). The reduction start required time threshold value is a threshold value for determining the measured SpO₂ to be determined as a stable state, a boundary state, and an exacerbation state. Here, the required time boundary threshold value and the required time exacerbation threshold value smaller than the required time boundary threshold value are determined. Determination is made as the stable state when the determined reduction start required time is greater than or equal to the required time boundary threshold value, determination is made as the boundary state when the determined reduction start required time is smaller than the required time boundary threshold value and greater than or equal to the required time exacerbation threshold value, and determination is made as the exacerbation state when the determined reduction start required time is smaller than the required time exacerbation threshold value.

The reduction start required time threshold value can be determined as a function of the exercise intensity, and in the present embodiment, is calculated by the following Equations 5 and 6, but this is not the sole case.

required time boundary threshold value=reference required time×0.85 [Equation 5]

required time exacerbation threshold value=reference required times×(100−(15+2 log₂ (load METs)))/100 [Equation 6]

The reference required time can be determined based on the exercise load. In the present embodiment, the reduction start required time determined based on the actual measurement by the walking performed at the time of diagnosis is the reference required time at the time of walking, and the exercise load of the determination target is also walking (3 METs), and hence the reference required time at the time of walking is used. For jogging or the like as well, actual measurement may be performed to store a reference index corresponding to the exercise load, and the reference required time may be selected according to the exercise load of the determination target.

The reference required time for other exercise loads may be calculated by one reference index, for example, calculation based on the reference required time at the time of walking. For example, it can be calculated based on the following Equation 7.

reference required time (load METs)=reference required time (reference METs)+log₃ (reference METs/load METs) [Equation 7]

Here, the reference required time=10:00 based on the actual measurement value at the time of diagnosis and the reference index and the determination target are both METs=3, and thus the required time boundary threshold value tt₁=8:30 and the required time exacerbation threshold value tt₂=8:11 are calculated based on Equations 5 and 6.

Whether or not the reduction start required time determined in S902 is greater than or equal to the required time boundary threshold value is determined (S906), where if true, determination is made as the stable state (S910), and if false, whether or not it is greater than or equal to the required time exacerbation threshold value is determined (S908). Determination is made as the boundary state if true (S912), and determination is made as the exacerbation state if false (S914).

Here, the determination target 1 is determined to be in the stable state as the reduction start required time t₁=9:00 and the required time boundary threshold value tt₁=8:30 or greater. The determination target 2 is determined to be in the boundary state as the reduction start required time t₂=8:15, the required time boundary threshold value tt₁=smaller than 8:30, and required time exacerbation threshold value tt₂=8:11 or greater. The determination target 3 is determined to be in the exacerbation state as the reduction start required time t₃=7:30, smaller than the required time exacerbation threshold value.

The fact that the reduction start required time of the determination target is shorter compared to the reference start required time means that the gas exchange preliminary function due to the disease is lowering, and the degree of progression of the disease can be determined based on the extent of shortness.

### Load Steady Saturation Degree

Next, an embodiment of a case where the load steady saturation degree is used as the reduction-related index will be described. A portion different from the example of the reduction integrated value and the reduction start required time will be described in detail, and the description of the similar portion will be omitted. As described above, the load steady saturation degree is the arterial oxygen saturation degree when a steady state is obtained after reduction in the arterial oxygen saturation degree is started.

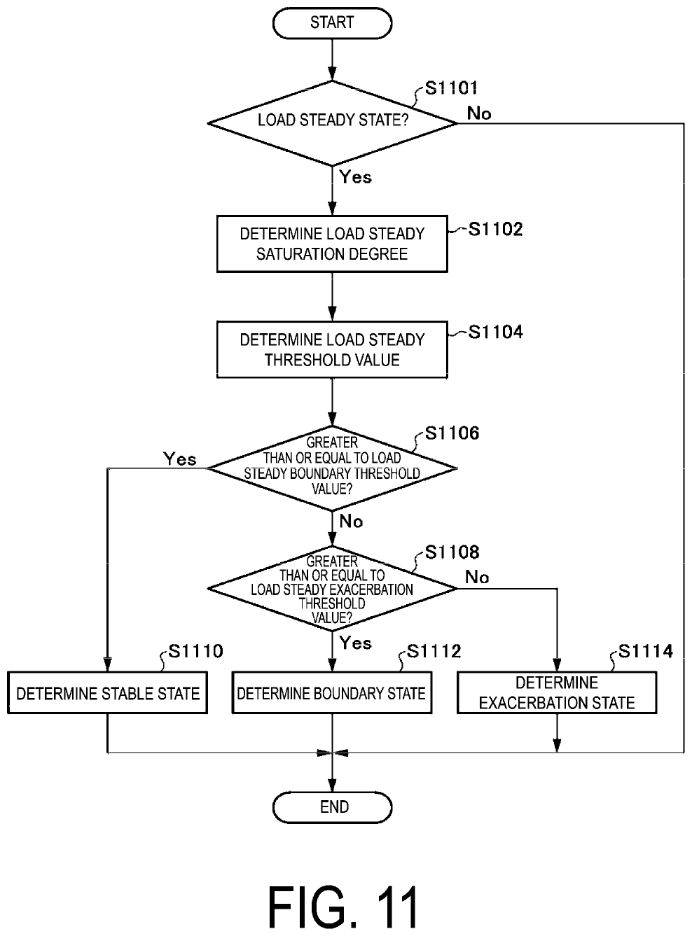

Of the progression degree determination processing flow illustrated in FIG. 3, a specific processing flow is illustrated in FIG. 11 for a case in which the load steady saturation degree is used as the reduction-related index in the reduction-related index determination and progression degree determination (S308).

First, whether or not the steady state is obtained after the SpO₂ becomes lower than the exercise load is determined based on the SpO₂ measurement values previously acquired in S304 (S1101). If not in the steady state, the reduction-related index determination and progression degree determination process are terminated, and the process returns to S304. If in the steady state, the load steady saturation degree is determined (S1102).

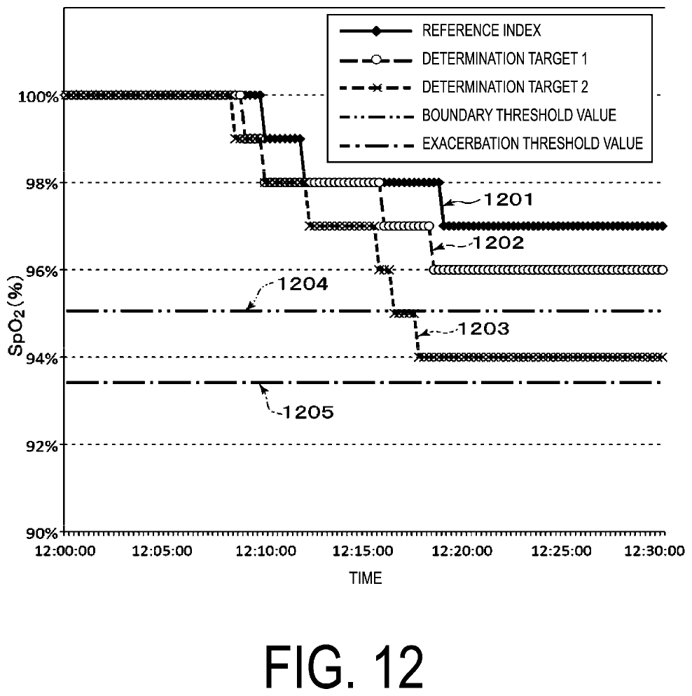

An example of the acquired SpO₂ measurement value to be determined is shown in FIG. 12. In FIG. 12, line 1201 indicates the transition of SpO₂ of the reference index, and lines 1202 and 1203 indicate the transition of the measured SpO₂ to be determined.

According to the measurement values of SpO₂ shown in FIG. 12, the reference index (1201) started to reduce at 12:10:00 and reached a steady state at 12:19:00. The load steady saturation degree is 97%. The determination target 1 (1202) started to reduce at 12:09:00 and reached a steady state at 12:18:30, and the load steady saturation degree is 96%. The determination target 2 (1203) started to reduce at 12:08:30 and reached a steady state at 12:17:00, and the load steady saturation degree is 94%.

Next, a load steady threshold value is determined (S1104). The load steady threshold value is a threshold value for determining the measured SpO₂ to be determined as a stable state, a boundary state, and an exacerbation state. Here, a load steady boundary threshold value and a load steady exacerbation threshold value smaller than the boundary threshold value are determined. Determination is made as the stable state when the determined load steady saturation degree is greater than or equal to the load steady boundary threshold value, determination is made as the boundary state when the determined load steady saturation degree is smaller than the load steady boundary threshold value and greater than or equal to the load steady exacerbation threshold value, and determination is made as the exacerbation state when the determined load steady saturation degree is smaller than the load steady exacerbation threshold value.

The load steady threshold value can be determined as a function of the exercise intensity, and in the present embodiment, is calculated by the following Equations 8 and 9, but this is not the sole case.

load steady boundary threshold=reference load steady saturation degree×0.98 [Equation 8]

load steady exacerbation threshold value=reference load steady saturation degree×(100−(2+log₂ (load METs)))/100 [Equation 9]

The reference load steady saturation degree can be determined based on the exercise load. In the present embodiment, the load steady saturation degree determined based on the actual measurement by the walking performed at the time of diagnosis is the reference load steady saturation degree at the time of walking, and the exercise load of the determination target is also walking (3 METs), and hence the reference load steady saturation degree at the time of walking is used. For jogging or the like as well, actual measurement may be performed to store a reference index corresponding to the exercise load, and the reference load steady saturation degree may be selected according to the exercise load of the determination target.

Furthermore, the reference load steady saturation degree for other exercise loads may be calculated by calculation based on the reference load steady saturation degree determined for one exercise load. For example, it can be calculated based on the following numeral 10.

reference load steady saturation degree (load METs)=reference load steady saturation degree (reference METs)+log₂ (reference METs/load METs) [Equation 10]

Here, the load steady saturation degree actually measured at the time of walking is defined as reference load steady saturation degree=97%, and both the reference index and the determination target have METs=3, and thus based on Equations 8 and 9, load steady boundary threshold value=95.06% (line 1204) and load steady exacerbation threshold value=93.42% (line 1205).

Whether or not the load steady saturation degree determined in S1106 is greater than or equal to the load steady boundary threshold value is determined, where if true, determination is made as the stable state (S1110), and if false, whether or not it is greater than or equal to the load steady exacerbation threshold value is determined (S1108). Determination is made as the boundary state if true (S1112), and determination is made as the exacerbation state if false (S114).

Since the determination target 1 has the load steady saturation degree of 96%, which is greater than or equal to the load steady boundary threshold value (95.06%), it is determined as the stable state, and since the determination target 2 has the load steady saturation degree of 94%, which is smaller than the load steady boundary threshold value (95.06%) and greater than or equal to the load steady exacerbation threshold value (93.42%), it is determined as the boundary state.

The fact that the load steady saturation degree of the determination target is reduced as compared to the reference load steady saturation degree means that the gas exchange preliminary function due to the disease is lowering, and the degree of progression of the disease can be determined based on the extent of reduction.

As a disease such as a respiratory disease or a circulatory disease progresses, the gas exchange preliminary function of the user (subject) with respect to the oxygen demand, which increases by exercise, lowers. The present invention makes it possible to determine the degree of progression of a disease by detecting the lowering in gas exchange preliminary function based on a reduction-related index by exercise load in the arterial oxygen saturation degree.

The manner and extent of reduction in the arterial oxygen saturation degree vary depending on the magnitude of the exercise load. In the embodiments of the present invention described above, the reduction-related index related to reduction by exercise load of the measured arterial oxygen saturation degree is determined based on the continuously measured arterial oxygen saturation degree, information indicating the magnitude of the exercise load is acquired, and the degree of progression of the disease is determined based on the information indicating the magnitude of the exercise load and the determined reduction-related index. By considering the magnitude of the exercise load, the degree of progression of the disease can be appropriately determined based on the reduction-related index related to the arterial oxygen saturation degree that reduces by the exercise load.

In each of the embodiment described above, the determination of the threshold value is executed in the determination process (S308), but may be determined when the reference index is acquired (S301). For example, in a case where it is determined to make a determination during walking, since the magnitude of the exercise load is determined, each threshold value can be determined at the stage of acquiring the reference index.

In the embodiment described above, the present invention is realized by using two devices of the arterial oxygen saturation degree measuring instrument 101 and the user device 102, but all functions may be realized by one device. For example, the user device 102 that can be worn by the user includes the arterial oxygen saturation degree measuring device 207 and the physical activity sensor 208, and can implement the function of the arterial oxygen saturation degree measuring instrument 101 described above. It can also be realized by having three or more devices implement the functions described above in a distributed manner.

The embodiments of the present invention have been described for illustrative purposes, but the present invention is not limited to these embodiments. The present invention may be implemented in various forms without departing from the scope thereof.

### REFERENCE SIGNS LIST

100: system
101: arterial oxygen saturation degree measuring instrument
102: user device
200: bus
201: processing device
202: display device
203: input device
204: storage device
205: communication device
206: program
207: arterial oxygen saturation degree measuring device
208: physical activity sensor
250: bus
251: processing device
252: output device
253: input device
254: storage device
255: communication device
256: program

1. A device for measuring a degree of progression of disease, the device comprising:
a processor that:
acquires a continuously measured arterial oxygen saturation degree,
determines a reduction-related index related to reduction by exercise load of the measured arterial oxygen saturation degree based on the continuously measured arterial oxygen saturation degree,
acquires information indicating a magnitude of the exercise load, and
determines the degree of progression of the disease based on the information indicating the magnitude of the exercise load and the determined reduction-related index.

2. The device according to claim 1, wherein
the reduction-related index includes a reduction integrated value based on a time integration of the arterial oxygen saturation degree from start of reduction of the arterial oxygen saturation degree to a steady state at the time of load, and
the processor:
determines a reduction integrated value of the measured arterial oxygen saturation degree based on the continuously measured arterial oxygen saturation degree, and
determines a degree of progression of a respiratory/circulatory disease based on the information indicating the magnitude of the exercise load and the determined reduction integrated value.

3. The device according to claim 2, wherein
the processor:
determines a reduction integrated boundary threshold value and a reduction integrated exacerbation threshold value smaller than the reduction integrated boundary threshold value based on the information indicating the magnitude of the exercise load,
determines a boundary state when the determined reduction integrated value is smaller than or equal to the reduction integrated boundary threshold value and greater than the reduction integrated exacerbation threshold value, and
determines an exacerbation state when the determined reduction integrated value is smaller than or equal to the reduction integrated exacerbation threshold value.

4. The device according to claim 3, wherein after the boundary state is determined, a newly measured arterial oxygen saturation degree is acquired, the determination is re-executed based on the acquired arterial oxygen saturation degree, and determination is made as the exacerbation state when the determination as the boundary state is repeated for greater than or equal to a predetermined number of times.

5. The device according to claim 1, wherein
the reduction-related index includes a reduction start required time from start of a load state by exercise until start of reduction of the arterial oxygen saturation degree, and
the processor:
acquires information indicating timing at which the load state by exercise has started,
determines a reduction start required time of the measured arterial oxygen saturation degree based on the timing at which the load state has started and the continuously measured arterial oxygen saturation degree, and
determines the degree of progression of the diseased based on the information indicating the magnitude of the exercise load and the determined reduction start required time.

6. The device according to claim 1, wherein
the reduction-related index includes a load steady saturation degree that is an arterial oxygen saturation degree when a steady state is obtained after reduction of the arterial oxygen saturation degree has started, and
the processor:
determines the load steady saturation degree based on the continuously measured arterial oxygen saturation degree, and
determines the degree of progression of the disease based on the information indicating the magnitude of the exercise load and the determined load steady saturation degree.

7. The device according to claim 1, wherein the processor presents warning information based on the determined degree of progression of the respiratory disease.

8. The device according to claim 1, wherein the arterial oxygen saturation degree is continuously measured and acquired.

9. The device according to claim 1, wherein movement of a user's body is detected to generate and acquire information indicating a magnitude of an exercise load.

10. A method for determining degree of progression of a disease, the method causing a processor to execute steps of:
acquiring a continuously measured arterial oxygen saturation degree;
determining a reduction-related index related to reduction by exercise load of the measured arterial oxygen saturation degree based on the continuously measured arterial oxygen saturation degree;
acquiring information indicating a magnitude of the exercise load; and
determining the degree of progression of the disease based on the information indicating the magnitude of the exercise load and the determined reduction-related index.

11. A non-transitory computer readable medium storing a program for causing a processor to execute the method described in claim 10.

12. A system for determining degree of progression of a disease, the system comprising:
a processor that:
continuously measures arterial oxygen saturation degree,
determines a reduction-related index related to reduction by exercise load of the measured arterial oxygen saturation degree based on the continuously measured arterial oxygen saturation degree,
acquires information indicating a magnitude of the exercise load, and
determines the degree of progression of the disease based on the information indicating the magnitude of the exercise load and the determined reduction-related index.
